(19) United States
(12) Patent Application Publication
(10) Pub. No.: US 2008/0050542 A1
(43) Pub. Date: Feb. 28, 2008

(54) Ink jet recording material

(75) Inventors: Satoshi Matsuura, Tokyo (JP); Kazuo Totani, Tokyo (JP)

Correspondence Address: DILWORTH & BARRESE, LLP, 333 EARLE OVINGTON BLVD., SUITE 702, UNIONDALE, NY, 11553, US

(73) Assignee: Oji Paper Co., Ltd., Tokyo (JP)

(21) Appl. No.: 11/894,295

(22) Filed: Aug. 21, 2007

(30) Foreign Application Priority Data
Aug. 23, 2006 (JP) 2006-226264

(51) Int. Cl. B41M 5/50; C07C 211/27; C09D 11/02

(52) U.S. Cl. 428/32.22; 106/31.6; 564/305

(57) ABSTRACT

An ink jet recording material free of blotting when printed in dye ink and pigment ink, superior in print density and permitting clear printing of a high-definition image is disclosed. The ink jet recording material comprises a substrate at least one surface of which is treated with an ink fixing agent, the ink fixing agent being a cationic surfactant represented by the following general formula:

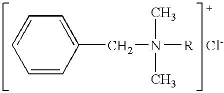

where R is an alkyl group having 8 to 18 carbon atoms.

### FIELD OF THE INVENTION

The present invention relates to an ink jet recording material not blotting when printed with dye ink or pigment ink, superior in print density and capable of printing high definition images clearly.

### BACKGROUND OF THE INVENTION

With the recent technical advance of ink jet printers for making hard copies at high speed, it has become possible to obtain clear images and high print quality. Accordingly, for further improving the print quality, there now exists a demand for a recording material of better quality. To meet this demand, various recording materials have been developed.

Aqueous inks for ink jet printers are classified into dye inks using dyes and pigment inks using pigments. The former is mainly used from the standpoint of clearness. Recently, however, the aqueous inks in question have come to be used also in printing large-sized posters for outdoor display. In this case, when a poster using dye ink is displayed outdoors over a long period, it is oxidized by ultraviolet light and ozone, resulting in that images formed using the dye ink become faded and poor-looking, thus giving rise to the drawback that a satisfactory print resistance to light is not obtained.

On the other hand, pigment inks are characteristic in that printed portions using the pigment inks are superior in storage stability. However, pigment particles of pigment inks are very large in comparison with the size of dyes used in dye inks, thus giving rise to the problem that the conventional ink jet recording materials for dye inks cannot afford clear print images.

Recently, the use of ink jet printers has been expanding from the conventional use of printing documents and the like to the use of wide advertisement such as posters. For printing advertisements or the like there usually are employed an ink jet printers able to effect printing on a wide printing material, which are called ink jet plotters for example. Printed matters obtained by using such ink jet plotters are displayed outdoors in many cases and therefore pigment inks superior in the storage stability of printed portions are mainly used. Besides, their use is for advertisement, it is necessary for images to be clearly visible even from a remote place. Consequently, there is an yearly tendency that the amount of ink discharged becomes larger.

Since dye inks and pigment inks are different in characteristics such as the pigment particles of pigment ink being very large in comparison with the dye size of dye inks, ink jet recording materials to be used exclusively for dye inks and pigment inks respectively have been provided as recording media. For example, in the case where a conventional ink jet recording material for dye inks is printed using pigment ink, there arise problems in practical use, for example, the pigment ink is not absorbed by the recording material, causing unevenness in printed portions, or the fixing of ink particles,is bad and ink peels off from printed portions when touched with a finger or the like.

Under the circumstances there has been a keen demand for the development of an ink jet recording material not blotting no matter which of dye ink and pigment ink may be used and having excellent printability.

To meet such a demand, as ink jet recording materials which exhibit excellent printability not matter which of dye inks and pigment inks may be used, there have been proposed a recording material using a partially hydrolyzed polyvinyl alcohol and an acrylic ester polymer as emulsion resin in an ink receptive layer (see, for example, JP 2000-309160A), a recording material containing in an ink receptive layer at least one emulsion type adhesive selected from acrylic polymers, ethylene-vinyl acetate polymers and vinyl acetate polymers (see, for example, JP 2005-280338A), a recording material containing a water-soluble metal salt in an ink receptive layer (see, for example, JP2002-274022A), and an ink jet recording sheet using base paper containing wood pulp and a filler as principal components, the base paper being surface-treated with a size press solution containing a cationic resin and a water-soluble polyvalent metal salt, the cationic resin being a polycondensate of three components which are dimethylamine, epichlorohydrin and amine, (see, for example, JP 2005-271522A).

However, none of them can be considered to fully satisfy the printability in the use of dye inks and pigment inks. Thus, at present, there still exists a keen demand for the development of an ink jet recording material which exhibits excellent printability no matter which of dye ink and pigment ink may be used.

### OBJECTS OF THE INVENTION

It is an object of the present invention to provide an ink jet recording material which exhibits excellent printability no matter which of dye ink and pigment ink may be used in printing the recording material.

### BRIEF SUMMARY OF THE INVENTION

Having made earnest studies so as to solve the above-mentioned problems, the present inventors have found out that the problems could be solved by adopting the following constructions.

According to the present invention there firstly is provided an ink jet recording material comprising a substrate, one or both surfaces of the substrate having a coating layer containing an ink fixing agent, characterized in that the ink fixing agent is a cationic surfactant represented by the following general formula:

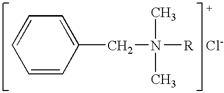

where R is an alkyl group having 8 to 18 carbon atoms.

According to the present invention there secondly is provided an ink jet recording material comprising a substrate, one or both surfaces of the substrate having a coating layer, the coating layer containing a pigment, an adhesive and an ink fixing agent, characterized in that the ink fixing agent is a cationic surfactant represented by the following general formula:

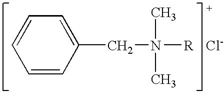

where R is an alkyl group having 8 to 18 alkyl groups.

According to the present invention there thirdly is provided the above second ink jet recording material wherein the coating layer contains 1 to 30 parts by mass of the cationic surfactant and 20 to 70 parts by mass of the adhesive, based on 100 parts by mass of the pigment.

According to the present invention there fourthly is provided the above second or third ink jet recording material wherein the adhesive contains a nonionic acrylic ester (co)polymer and a cationic acrylic ester (co)polymer.

According to the present invention there fifthly is provided the above fourth ink jet recording material wherein the nonionic acrylic ester (co)polymer is coated with a water-soluble polymer protective colloid.

According to the present invention there sixthly is provided an ink jet recording material wherein the water-soluble polymer protective colloid is polyvinyl alcohol.

According to the present invention there seventhly is provided the above fourth ink jet recording material wherein the substrate is a gas-permeable substrate.

According to the present invention there eighthly is provided an ink jet recording material wherein the substrate is a gas-impermeable substrate.

### EFFECT OF THE INVENTION

According to the present invention it is possible to provide an ink jet recording material exhibiting excellent blotting-free printability no matter which of dye ink and pigment ink may be used in printing the recording material superior in ink density.

### DETAILED DESCRIPTION OF THE INVENTION

The present invention will be described in detail hereinunder.

In connection with the ink jet recording material of the present invention, by the phrase “one or both surfaces of the substrate having a coating layer containing an ink fixing agent” it is meant that a treatment has been performed so as to let a cationic surfactant of the following general formula be present on at least one surface of the substrate of the ink jet recording material. Layer construction and coating layer forming method are not specially limited. For example, a coating solution containing the cationic surfactant may be applied onto one or both surfaces of the surface of the substrate. The coating solution to be applied onto the substrate may be a mixture with other substances. The substrate may be impregnated with the coating solution. Further, at the time of forming the substrate such as paper, the cationic surfactant may be added thereto. The cationic surfactant may be applied to the substrate by suitable means according to the type and properties of the substrate used.

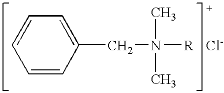

where R is an alkyl group having 8 to 18 carbon atoms.

### (Layer Construction)

The present invention resides in an ink jet recording material comprising a substrate, at least one surface of the substrate having an ink fixing agent-containing coating layer thereon, characterized in that the ink fixing agent is a cationic surfactant represented by the above general formula.

Such a coating layer may be provided on not only one surface but also both surfaces of the substrate. With the coating layers on both surfaces of the substrate, it becomes possible to effect clear prints on both surfaces of the ink jet recording material.

The coating layer on each surface may comprise plural layers. An undercoating layer may be provided between the substrate and the coating layer. Further, an overcoating layer may be provided on the coating layer in order to impart gloss to the recording material or enhance the storage stability thereof insofar as the printability of the coating layer is not impaired.

### (Substrate)

The substrate may be a gas-permeable substrate or a gas-impermeable substrate. Both may be used selectively according to use and the purpose of use. A gas-permeable substrate is preferred in order to separate the pigment in pigment ink and solvent as soon as possible and enhance the pigment fixing property and print density.

### (Gas-Permeable Substrate)

Suitable examples of gas-permeable substrates are wood-free paper, art paper, on-machine coated paper, cast-coated paper, kraft paper, baryta paper, saturating paper, metallized paper, and water-soluble paper.

A gas-permeable substrate is mainly composed of wood pump and, if necessary, a filler. Examples of employable wood pulps include various chemical pulps, mechanical pulp and regenerated pulp. As to these pulps, the degree of beating (freeness) can be adjusted by a beater in order to adjust the paper stiffness and paper making adaptability. The degree of bearing (freeness) of the pulp used is not specially limited, but is generally in the range of 250 to 550 ml (CSF: JIS P8121). For improving smoothness it is desirable to enhance the degree of beating. As to unevenness of paper and blur of a recorded image, which are caused upon printing on paper by moisture contained in ink, it is, for obtaining a good result, better not to enhance the degree of beating in many cases. Therefore, the freeness of about 300 to 500 ml (CSF: JIS P8121) is preferred.

The filler is a component which is used for the purpose of imparting opacity to the substrate or adjusting the ink absorbability. As the filler there may be used, for example, calcium carbonate, calcined kaolin, silica, or titanium oxide. Particularly, the use of calcium carbonate is preferred because it affords a substrate having a high degree of whiteness, with the result that glossy impression of the ink jet recording material is enhanced. The content (ash content) of the filler in the paper substrate is preferably 1 to 20 mass %. If the filler content is too high, there is a fear that the paper stiffness may be deteriorated. If the filler content is too low, the gas permeability of the paper substrate will be deteriorated. A preferred filler content is 7 to 20 mass %. As long as the filler content falls under this range, the smoothness, gas permeability and paper stiffness are balanced, so that an ink jet recording material superior in smooth impression becomes easier to be obtained.

For example, size, fixing agent, paper stiffness enhancing agent, yield improving agent, dye and fluorescent whitener may be added as aids to the gas-permeable substrate. Further, in a size press process in a paper making machine, for example starch, polyvinyl alcohol and cation resin may be applied to or impregnated into the substrate to adjust the surface strength and the sizing degree. Stockigt sizing degree (as 100 g/m² paper) is preferably 1 to 200 seconds. If the sizing degree high, it is impossible to separate pigment ink and solvent quickly from each other and ink absorbability and print density may be deteriorated. A more preferred range of the sizing degree is 5 to 120 seconds. The weight of the substrate is not specially limited, but is usually in the range of 20 to 400 g/m², preferably 50 to 250 g/m², most preferably 60 to 200 g/m².

### (Gas-Impermeable Substrate)

Examples of employable gas-impermeable substrates are polymer films (including those called synthetic paper) such as films of polyethylene, polypropylene, soft polyvinyl chloride, rigid polyvinyl chloride, and polyester, metallic films, resin-coated paper comprising paper or nonwoven fabric and thermoplastic resins laminated thereto, the paper being for example, wood-free paper, art paper, on-machine coated paper, cast-coated paper, foil paper, kraft paper, saturating paper, metallized paper or water-soluble paper, and laminated sheets comprising paper or nonwoven fabric and films laminated thereto. As preferred examples of substrates there are mentioned so-called synthetic paper prepared by stretching polypropylene and subjecting the stretched polypropylene to a special processing, which are typified by YUPO (trade name, a product of YUPO Corp.), and resin-coated paper comprising a paper substrate and a polyolefin fin (preferably polyethylene resin) laminated thereto. In the case of a gas-impermeable substrate, the dye, pigment—solvent separation speed in ink is low, but the ink solvent does not penetrate into the substrate and therefore the use of a gas-impermeable substrate is effective in uses wherein cockling poses a problem.

Synthetic paper is usually prepared by extruding polypropylene resin containing an inorganic pigment such as calcium carbonate and biaxially stretching the extruded resin to form voids in the interior thereof. It is preferably a laminated sheet comprising plural layers. Particularly, it is preferable to use synthetic paper having an unevenness-free skin layer on its surface on which a recording layer is formed. On the synthetic paper surface there may be formed various layers, e.g., anchor layer, primer layer and antistatic layer, for improving coatability or for improving antistatic property.

As resin-coated paper, a substrate produced by coating polyethylene resin with titanium oxide kneaded therein onto a paper surface is particularly preferred because its finished appearance is almost equal to that of photographic printing paper. The thickness of the polyethylene resin layer is preferably 3 to 50 μm, more preferably 5 to 30 μm. If the thickness of the polyethylene resin layer is smaller than 3 μm, defects such as holes in the polyethylene resin are apt to increase at the time of resin coating and it is in many cases difficult to control the thickness. It is also difficult to attain smoothness. Conversely, if the polyethylene resin thickness exceeds 50 μm, the resulting effect is not so outstanding despite of an increase of cost, which is uneconomical.

The paper base used in the resin-coated paper is produced using wood pulp as a main component. As the wood pulp there may be used any of various chemical pulps, mechanical pulp and regenerated pulp. As to these pulps, the degree of beating (freeness) can be adjusted by a beater in order to adjust paper stiffness, smoothness and paper making adaptability. The degree of beating (freeness) is not particularly limited, but is preferably in the range of 250 to 550 ml (CSF: JIS P8121). Chlorine-free pulps such as ECF and TCF are also employable preferably. Where required, a pigment may be added. As preferred examples of pigments there are mentioned talc, calcium carbonate, clay, kaolin, calcined kaolin, silica, and zeolite. By the addition of a pigment it is possible to enhance the opacity and smoothness. If the pigment is added to excess, the paper stiffness may be deteriorated. The amount of the pigment to be added is preferably in the range of 1 to 20 mass % based on the amount of the wood pulp used.

For the purpose of improving the adherence between the substrate and the coating layer, the coating layer-forming surface of the substrate may be subjected beforehand to an adherence or bonding improving treatment. Particularly, in case of using resin-coated paper as the substrate, it is preferable apply a corona discharge treatment to the surface of the resin-coated layer or form an undercoating layer on the resin-coated layer surface with use of gelatin or polyvinyl alcohol.

### (Ink Fixing Agent)

In the ink jet recording material of the present invention a cationic surfactant represented by the following general formula is used as an ink fixing agent. As to the details of the mechanism which permits the cationic surfactant of the following general formula to contribute to imparting excellent printability to the recording material, it relies on future studies. However, it is presumed that since the cationic surfactant is high in its reaction rate with ink colorant, the ink colorant can be fixed to the surface without dropping into the layer and that therefore excellent printability is attained.

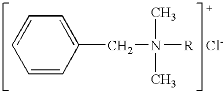

where R is an alkyl group having 8 to 18 carbon atoms which alkyl group may be a straight-chain or branched alkyl group.

As examples of the cationic surfactant used in the ink jet recording material of the present invention there are mentioned decyldimethylbenzylammonium chloride, dodecyldimethylbenzylammonium chloride, tetradecyldimethylbenzylammonium chloride, hexadecyldimethylbenzylammonium chloride, octadecyldimethylbenzylammonium chloride, and isooctyldimethylbenzylammonium chloride, with dodecyldimethylbenzylammonium chloride and tetradecyldimethylbenzylammonium chloride being most preferred in point of printability.

The amount of the ink fixing agent is preferably 5 to 90 parts by mass, more preferably 10 to 80 parts by mass, based on 100 parts by mass of the coating layer which contains the ink fixing agent. If the amount of the ink fixing agent is smaller than 5 parts by mass, there is a fear that blotting may become conspicuous, and if it exceeds 90 parts by mass, there is a fear that the foamability of the coating solution may be deteriorated. In case of using a pigment, the amount of the ink fixing agent is preferably 1 to 30 parts by mass, more preferably 3 to 20 parts by weight, based on 100 parts by mass of the pigment. If the amount of the ink fixing agent is smaller than 1 part by mass, there is a fear that blotting may become conspicuous and print density may be deteriorated, and if it exceeds 30 parts by mass, there is a fear that the foamability of the coating solution may be deteriorated, with consequent occurrence of white portions on a coated surface due to bubbles and deterioration of print density.

Other than the ink fixing agent of the foregoing general formula there may be used a known ink fixing agent insofar as the function and effect of the ink fixing agent defined in the present invention are not impaired. As examples of known ink fixing agents there are mentioned the following commercially available ones: 1) polyalkyleneamines such as polyethyleneamine and polypropylene, or derivatives thereof, 2) acrylic resins having tertiary or quaternary ammonium groups, 4) dicyan-based cationic resins typified by dicyandiamide-formalin polycondensates, 5) polyamine-based cation resins typified by dicyandiamide-diethylenetriamine polycondensates, 6) dimethylamine-epichlorohydrin addition polymers, 7) diallyldimethylammonium chloride-SO₂ copolymers, 8) diallylamine salt-SO₂ copolymers, 9) dimethyldiallylammonium chloride polymers, 10) allylamine salt polymers, 11) homo- or copolymers of vinylbenzyltriallylammonium salt, 12) dialkylaminoethyl (meth)acrylate quaternary salt copolymers, 13) acrylamide-diallylamine salt copolymers, 14) aluminum salts such as polyaluminum chloride and polyaluminum acetate. These compounds may be used each alone or several of them may be used in combination.

### (Pigment in Coating Layer)

In case of using a pigment, any pigment may be used insofar as it has heretofore been used in an ink receptive layer of an ink jet recording material. Examples of employable pigments are inorganic pigments such as light calcium carbonate, heavy calcium carbonate, kaolin, talc, calcium sulfate, titanium dioxide, zinc oxide, zinc sulfide, zinc carbonate, satin white, aluminum silicate, diatomaceous earth, calcium silicate, magnesium silicate, aluminum hydroxide, alumina, pseudoboehmite, lithopone, zeolite, hydrated halloysite, magnesium carbonate and magnesium hydroxide, and organic pigments constituted by such resins as acrylic or methacrylic resins, polyvinyl chloride resins, polyvinyl acetate resins, polyester resins, styrene-acryl resins, styrene-butadiene resins, styrene-isoprene resins, polycarbonate resins, silicone resins, urea resins, melamine resins, epoxy resins, phenolic resins, and diallyl phthalate resins. These pigments may each be in a truly spherical shape or amorphous. Likewise, they may be porous or non-porous. They each may be used alone or two or more of them may be used in combination. Amorphous silica is more preferred. How to prepare silica is not specially limited. Silica produced by any of arc process, dry process and wet process (precipitation or gelling method) is employable in the present invention.

An average particle diameter of secondary particles of silica is preferably 2 to 12 μm, more preferably 4 to 8 μm. If the average particle diameter is smaller than 2 μm, then when such silica is used in an ink jet recording sheet for dye ink, the absorbability of the sheet for the dye ink will be deteriorated and so will be the coating strength. On the other hand, if the average particle diameter of secondary particles of silica exceeds 12 μm, both ink jet recording sheet for dye ink and ink jet recording sheet for pigment ink encounter a problem that unevenness in print is apt to occur. The average particle diameter of silica is measured by the coulter counter method. According to this method, silica is dispersed ultrasonically for 30 seconds in distilled water and the thus-dispersed silica is used as a sample for the measurement. It represents a volume average particle diameter.

In case of using a pigment, the amount of the pigment is 50 to 85 parts by mass, preferably 60 to 70 parts by mass, based on 100 parts by mass of the coating layer which contains the ink fixing agent and the pigment. If the amount of the pigment is smaller than 50 parts by mass, there is a fear that print density may be deteriorated, and if it is larger than 85 parts by mass, there is a fear that the coating strength may be deteriorated.

### (Adhesive in Coating Layer)

The adhesive used in the coating layer is not specially limited, but there may be used conventional known adhesives generally used for ink jet recording materials. Examples are proteins such as casein, soy protein and synthetic protein; various starches such as starch and oxidized starch; polyvinyl alcohol and derivatives thereof; cellulose derivatives such as carboxymethyl cellulose and methyl cellulose; conjugated diene resins such as styrene-butadiene resin and methyl methacrylate-butadiene copolymer; acrylic resins such as polymers or copolymers of acrylic acid, methacrylic acid, acrylic ester and methacrylic ester; and vinyl resin such as ethylene-vinyl acetate copolymer. These may be used each along or two or more thereof may be used in combination.

Particularly preferred are acrylic ester (co)polymers each containing a nonionic acrylic ester (co)copolymer and a cationic acrylic ester (co)polymer, the nonionic acrylic ester (co)polymer being coated with a water-soluble polymer protective colloid, the water-soluble polymer being polyvinyl alcohol.

As adhesives there generally are known water-soluble adhesives and emulsion type adhesives, but it has turned out that by using a water-soluble polymer protective colloid type acrylic ester copolymer there is attained excellent printability no matter which of dye ink and pigment ink may be used. A combined use thereof with a cationic acrylic ester (co)polymer is particularly preferred. As to by what mechanism the water-soluble protective colloid type acrylic ester (co)polymer as an adhesive contributes to the excellent printability, the details thereof is not certain. However, it is presumed that since the acrylic ester (co)polymer is coated with a water-soluble polymer, the ink colorant adheres or fixed to the surface of the water-soluble polymer which is a protective colloid and the ink particles or colorant of dye ink or pigment ink is present in the acrylic ester (co)polymer without being hidden, resulting in excellent color developability being attained.

The proportion of the adhesive is preferably 10 to 95 parts by mass, more preferably 20 to 90 parts by mass, based on 100 parts by mass of the coating layer which contains the ink fixing agent. If the amount of the adhesive is smaller than 10 parts by mass, it is difficult to increase the amount of the coating solution, and if it is larger than 90 parts by mass, there is a fear of ink overflow and blotting becoming more conspicuous. In case of using a pigment, the proportion of the adhesive is 20 to 70 parts by mass, more preferably 40 to 60 parts by mass, based on 100 parts by mass of the pigment. If the amount of the adhesive exceeds 70 parts by mass, there is a fear of ink overflow and the occurrence of blotting, and if the amount of the adhesive is smaller than 20 parts by mass, the strength of the coating layer tends to become lower.

To the coating layer there may be suitably added various aids which are generally used in the production of coated paper such as, for example, thickener, defoaming agent, wetting agent, surfactant, colorant, antistatic agent, light resistance aid, ultraviolet absorber, antioxidant, and atiseptic.

The amount of the coating solution applied for the coating layer containing the ink fixing agent is not specially limited, but is preferably 0.1 to 30 g m², more preferably 0.5 to 20 g/m². If the amount of the coating solution applied is smaller than 0.1 g/m², ink absorbability and image clearness are apt to be deteriorated, and if the amount of the coating solution applied exceeds 30 g/m², the coating strength and image clearness are apt to be deteriorated. The coating layer may be a laminate of plural coating layers. In this case, the coating layers may be of different compositions.

The coating layer can be formed using any of various known coaters, including blade coater, air knife coater, roll coater, bar coater, gravure coater, rod blade coater, lip coater, curtain coater, die coater, reverse roll coater, kiss roll coater, cast coater, champlex coater, brush coater, gate roll coater, hamilton coater, KCM coater, size press coater, metered size press, metered film transfer roll coater, and slide bead coater.

Drying conditions for the coating layer are adjusted for example by adjusting the concentration of the coating solution. In this connection, the behavior of the absorbing speed varies also depending on drying conditions. It is preferable to dry the coating solution under as strong drying conditions as possible. However, excessive drying tends to lead to deterioration of the color developability.

The coating operation may be followed by a finishing process using a calender such as, for example, machine calender, super calender, or soft calender. However, also as to such a finish process, it is necessary to make adjustment within the range not impairing printability.

The present invention will be described concretely by way of working examples thereof, but it goes without saying that the present invention is not limited by the following Examples. Unless otherwise mentioned, parts and % in the Examples are of solids exclusive of water and represent parts by mass and mass %, respectively.

### EXAMPLE 1

### [Preparing a Coating Solution for Coating Layer]

100 parts of wet silica (trade name: Fine Seal X-60, a product of Tokuyama Corp.) as a pigment, 10 parts of tetradecyldimethylbenzylammonium chloride (trade name: M2-100R, a product of NOF CORP.) as an ink fixing agent, and 20 parts of silyl-modified polyvinyl alcohol (PVA) (trade name: R-1130, a product of Kuraray Co., Ltd.), 15 parts of a water-soluble polymer protective colloid type nonionic acrylic ester copolymer (trade name: VINYBLAN 2680, a product of Nisshin Chemical Co., Ltd.), and 10 parts of a cationic acrylic ester copolymer (trade name: VINYBLAN 2647, a product of Nisshin Chemical Co., Ltd.), as adhesives, were mixed and dispersed to prepare a coating solution having a solids content of 20%.

### [Preparing an Ink Jet Recording Material]

The coating solution was applied in an amount of 10 g/m² onto one surface of wood-free paper having a weight of 170 g/m² and then dried to prepare an ink jet recording material.

### EXAMPLE 2

An ink jet recording material was prepared in the same way as in Example 1 except that the ink fixing agent, tetradecyldimethylbenzylammonium chloride (trade name: M2-100R, a product of NOF CORP.), contained in the coating solution prepared in Example 1 was substituted by dodecyldimethylbenzylammonium chloride 60%/tetradecylbenzyl ammonium chloride 40% (trade name: F2-50R, a product of NOF CORP.).

### EXAMPLE 3

An ink jet recording material was prepared in the same way as in Example 1 except that 15 parts of the water-soluble polymer protective colloid type nonionic acrylic ester copolymer (trade name: VINYBLAN 2680, a product of Nisshin Chemical Co., Ltd.) and 10 parts of the cationic acrylic ester copolymer (trade name: VINYBLAN 2647, a product of Nisshin Chemical Co., Ltd.) used in preparing the coating solution in Example 1, were substituted by 25 parts of polyvinyl alcohol (trade name: PVA-117, a product of Kuraray Co., Ltd.).

### EXAMPLE 4

An ink jet recording material was prepared in the same way as in Example 1 except that 100 parts of the wet silica (trade name: Fine Seal X-60, a product of Tokuyama Corp.) used in preparing the coating solution in Example 1 was substituted by 100 parts of light calcium carbonate (trade name: ED-111, a product of Yonesho Lime Industry Co., Ltd.).

### EXAMPLE 5

A mixed coating solution consisting of 20 parts of dodecyldimethylbenzylammonium chloride 60%/tetradecylbenzyl ammonium chloride 40% (trade name: F2-50R, a product of NOF CORP.) as an ink fixing agent and 30 parts of oxidized starch (trade name: Ace A, a product of Ohji Corn Starch) was applied in an amount of 5 g/m² to one surface of wood-free paper having a weight of 170 g/m² by means of a two-roll size press, then dried to afford an ink jet recording material.

### COMPARATIVE EXAMPLE 1

An ink jet recording material was prepared in the same way as in Example 1 except that the ink fixing agent, tetradecyldimethylbenzylammonium chloride (trade name: M2-100R, a product of NOF CORP.), contained in the coating solution prepared in Example 1 was substituted by polydiallyldimethylammonium chloride (trade name: UNISENCE CP-91, a product of SENKA CORP.).

### COMPARATIVE EXAMPLE 2

An ink jet recording material was prepared in the same way as in Example 1 except that the ink fixing agent, tetradecyldimethylbenzylammonium chloride (trade name: M2-100R, a product of NOF CORP.), contained in the coating solution prepared in Example 1 was substituted by a dicyandiamide-polyallylamine copolymer (trade name: Neofix E-117, a product of Nicca Chemical).

### COMPARATIVE EXAMPLE 3

An ink jet recording material was prepared in the same way as in Example 5 except that 20 parts of the ink fixing agent, dodecyldimethylbenzylammonium chloride 60%/tetradecyldimethylbenzylammonium chloride 40% (trade name: F2-50R, a product of NOF CORP.) used in Example 5 was substituted by polydiallyldimethylammonium chloride (trade name: UNISENCE CP-91, a product of SENKA CORP.).

The ink jet recording materials prepared in the above Examples and Comparative Examples were evaluated for print density, blotting and print water resistance by the following methods.

In making evaluation, printing to the ink jet recording materials was performed using a commercially available pigment ink jet printer (trade name: Image PROGRAF W6200, a product of CANON INC., printing mode: Thick Coated Paper/Clear) and a commercially available dye ink jet printer (trade name: PIXUS ip8600, a product of CANON INC., printing mode: Mat Photo Paper/Clear).

### [Print Density]

An image (“High Definition Color Digital Standard Image XYZ/JIS-SCID,” Identification Mark: S6, Image Name: Color Chart) published by a foundation, Japanese Standards Association, was printed on two models of printers, i.e., ImagePROGRAF W6200 (using pigment ink) and PIXUS ip8600 (using dye ink), and a highest tone portion of black was measured for print density using RD-914 (a product of Guretag Macbeth Co.).

### [Blotting]

Print blotting from a print boundary portion on the two printers, ImagePROGRAF W6200 and PIXUS ip8600 was visually evaluated.

TABLE 1

| | |
|---|---|
| W6200 | ip8600 |
| (Pigment Ink) | (Dye Ink) |

| | Bk Print | | Bk Print | |
|---|---|---|---|---|
| | Density | Blotting | Density | Blotting |
| Example 1 | 1.56 | ◯ | 1.74 | ⊚ |
| Example 2 | 1.60 | ⊚ | 1.78 | ⊚ |
| Example 3 | 1.53 | ◯ | 1.70 | ◯ |
| Example 4 | 1.49 | ◯ | 1.68 | ◯ |
| Example 5 | 1.48 | ◯ | 1.66 | ◯ |
| Comparative | 1.41 | X | 1.59 | Δ |
| Example 1 | | | | |
| Comparative | 1.40 | Δ | 1.55 | X |
| Example 2 | | | | |
| Comparative | 1.32 | X | 1.45 | X |
| Example 3 | | | | |

⊚: Excellent level, with no print blotting recognized.
◯: Print somewhat blots, but poses no problem in practical use.
Δ: Print somewhat blots and somewhat poses a problem in practical use.
X: Marked print blotting, posing a serious problem in practical use.

As is apparent from Table 1, the ink jet recording materials according to the present invention exhibited little blotting with respect to images printed using dye ink and pigment ink and are superior in print density.

### INDUSTRIAL APPLICABILITY

According to the present invention, as set forth above, it is possible to provide an ink jet recording material free of blotting and superior in print density when printed using dye ink and pigment ink and permitting clear printing of a high-definition image. Thus, the ink jet recording material of the present invention is extremely useful in practical use.

What is claimed is:

1. An ink jet recording material comprising a substrate, one or both surfaces of said substrate having a coating layer containing an ink fixing agent, characterized in that said ink fixing agent is a cationic surfactant represented by the following general formula: 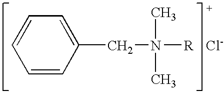
where R is an alkyl group having 8 to 18 carbon atoms.

2. An ink jet recording material comprising a substrate, one or both surfaces of said substrate having a coating layer, said coating layer containing a pigment, an adhesive and an ink fixing agent, characterized in that said ink fixing agent is a cationic surfactant represented by the following general formula: 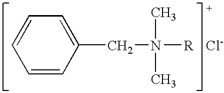
where R is an alkyl group having 8 to 18 carbon atoms.

3. An ink jet recording material according to claim 2, wherein said coating layer contains 1 to 30 parts by mass of said cationic surfactant and 20 to 70 parts by mass of said adhesive, based on 100 parts by mass of said pigment.

4. An ink jet recording material according to claim 2, wherein said adhesive contains a nonionic acrylic ester polymer and a cationic acrylic ester polymer.

5. An ink jet recording material according to claim 4, wherein said nonionic acrylic ester polymer is coated with a water-soluble polymer protective colloid.

6. An ink jet recording material according to claim 5, wherein said water-soluble polymer protective colloid is polyvinyl alcohol.

7. An ink jet recording material according to claim 1, wherein said substrate is a gas-permeable substrate.

8. An ink jet recording material according to claim 1, wherein said substrate is a gas-impermeable substrate.
